(19) United States
(12) Patent Application Publication
(10) Pub. No.: US 2010/0105934 A1
(43) Pub. Date: Apr. 29, 2010

(54) FULVESTRANT INTERMEDIATE

(76) Inventors: Gerrit J. B. Ettema, Beuningen (NL); Reinerus G. Gieling, Beuningen (NL)

Correspondence Address: SYNTHON IP INC, P.O. BOX 1364, FAIRFAX, VA, 22038-1364, US

(21) Appl. No.: 12/579,478

(22) Filed: Oct. 15, 2009

Related U.S. Application Data
(60) Provisional application No. 61/105,626, filed on Oct. 15, 2008.

(51) Int. Cl. C07J 1/00

(52) U.S. Cl. 552/626; 552/628

(57) ABSTRACT

A compound of formula (1)

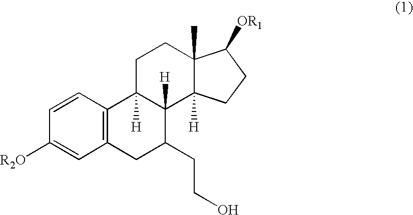

wherein R₁ represents hydrogen or an acetyl group and R₂ represents a methyl, acetyl, or benzyl group, is useful as an intermediate in making fulvestrant. The compound (1) can be isolated as a crystalline material and can be provided in a ratio of 7-alpha epimer to 7-beta epimer in the range of 95:5 to 100:0.

This application claims the benefit of priority under 35 U.S.C. §119(e) from U.S. provisional application Ser. No. 61/105,626, filed Oct. 15, 2008; the entire contents of which are incorporated herein by reference.

### BACKGROUND OF THE INVENTION

The present invention relates to processes for making fulvestrant and to intermediates useful therein. Fulvestrant is the generic name for (7alpha,17beta)-7-(9-((4,4,5,5,5-pentafluoropentyl)sulfinyl)nonyl-estra-1,3,5(10)-triene-3,17-diol having the following formula (A).

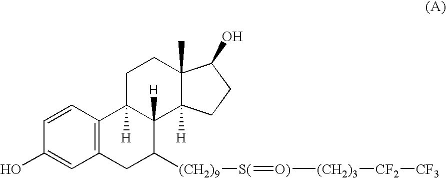

It is a 7alpha-substituted analogue of estradiol belonging to a class of agents known as selective estrogen receptor down-regulators (SERDs); i.e., an estrogen receptor antagonist without known agonist effects. Fulvestrant has been approved for the treatment of hormone receptor positive metastatic breast cancer in postmenopausal women with disease progression following antiestrogen therapy as a once-monthly injectable under the brand name FASLODEX® (AstraZeneca). Fulvestrant has been disclosed in EP 0138504 (U.S. Pat. No. 4,659,516).

In essence, the fulvestrant molecule consists of two parts: the steroidal skeleton (estra-1,3,5(10)-triene-3,17β-diol) and a long side chain linked to the skeleton in the 7-position. As the carbon in the 7-position is chiral, the side chain may be linked to the steroidal skeleton in two epimeric conformations. For pharmaceutical effect, the side chain must be linked solely in the alpha-direction (R-conformation).

Various synthetic routes are known in the prior art which comprise coupling a long side chain the 7-position of a properly modified steroidal skeleton; sometimes in whole and sometimes in parts. Known key coupling reactions on the position 7 within the fulvestrant synthesis comprise:

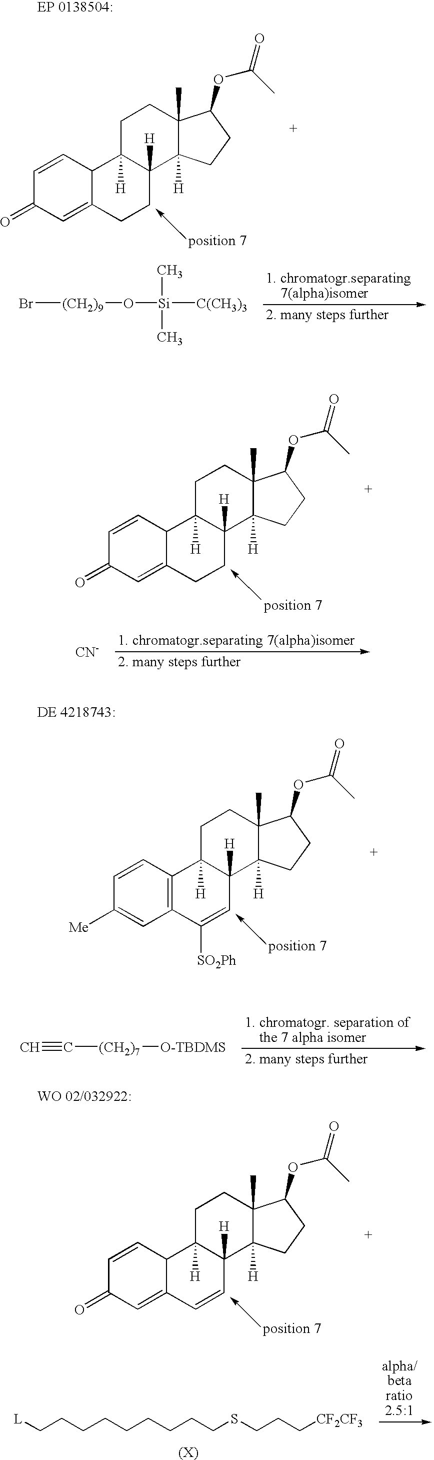

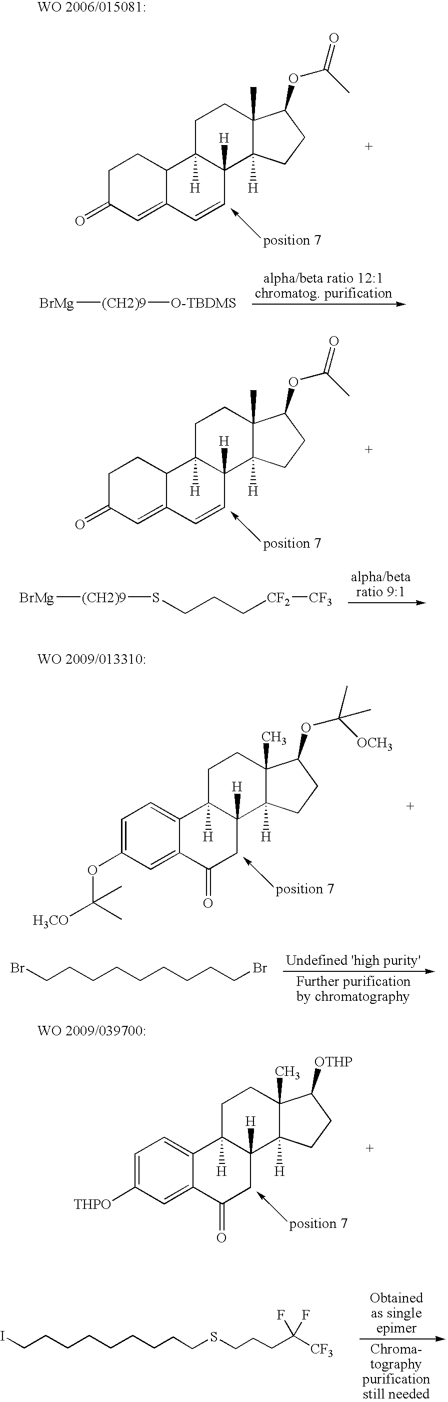

In most of the known routes, the stereoselectivity of the coupling on the position 7 is incomplete and the only known way of purification with the aim to isolate the correct conformer of the coupled product is by column chromatography. Crystallization as a tool for obtaining the correct diastereomer is possible only in later stages of the synthesis. Furthermore, the alkylation may also proceed at other reactive sites of the steroidal skeleton (e.g. on the C═O oxygen of the compounds of WO 2009/039700).

Thus, there is a need to improve the synthesis of fulvestrant; particularly it would be advantageous to provide a crystalline 7-substituted intermediate with a high degree of the epimeric purity.

### SUMMARY OF THE INVENTION

The present invention relates to a useful intermediate compound in making fulvestrant and related compounds. Accordingly a first aspect of the invention relates to a compound of the formula (1)

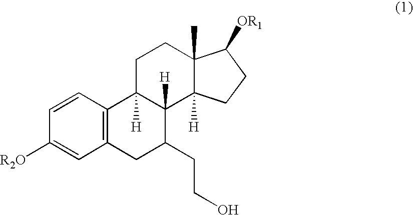

wherein R₁ is hydrogen or an acetyl group and R₂ is methyl, acetyl or benzyl group. The compound of formula (1) can be formed with a high ratio of the 7-alpha epimer relative to the 7-beta epimer and furthermore crystallization thereof serves to enhance the 7-alpha epimer content. The ratio of 7-alpha:7 beta epimers can be in the range of 95:5 to 100:0 and even 99:1 to 100:0 (i.e., at least 99% 7-alpha epimeric purity). The ability to obtain the compound of formula (1) in a solid, especially crystalline, form is also advantageous. Compounds of formula (1) are thus useful intermediates in making fulvestrant and related compounds.

Another aspect of the invention relates to a process, which comprises crystallizing an epimerically impure compound of formula (1):

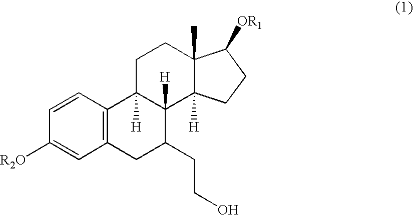

wherein R₁ represents hydrogen or an acetyl group and R₂ represents a methyl, acetyl, or benzyl group, to form an epimerically purified compound of formula (1) in the form of a crystalline material. Generally the epimerically impure compound of formula (1) has a 7-alpha epimeric purity of 75% to 90%, while the epimerically purified compound can have a 7-alpha epimeric purity of at least 95%.

A further aspect of the invention relates to a process for making fulvestrant, which comprises:

(i) providing a compound of formula (1) having a 7-alpha epimeric purity of at least 95%

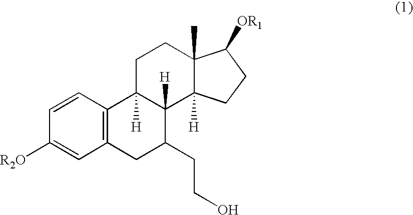

wherein R₁ represents hydrogen or an acetyl group and R₂ represents a methyl, acetyl, or benzyl group; and

(ii) converting said compound of formula (1) to a compound of the formula (A)

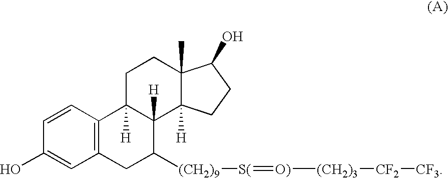

The converting is typically performed in several steps. In a common embodiment the converting comprises: (a) reacting the compound of formula (1) with a donor leaving group to form a compound of formula (2)

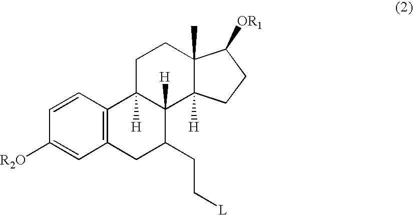

wherein L represents a leaving group such as a halogen or a sulfonyloxy group; and then (b) transforming the compound of formula (2) into the compound of formula (A) (in one or more steps).

An additional aspect of the present invention thus relates to a compound of formula (2)

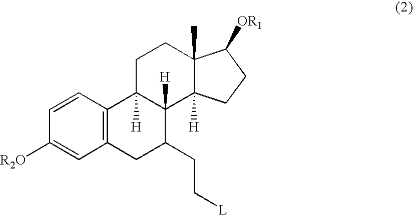

wherein R₁ and R₂ have the same meaning as above and L is a leaving group such as halogen, preferably bromine, an alkylsulfonyloxy group, preferably methane sulfonyloxy group, an arylsulfonyloxy group, preferably benzene sulfonyloxy- or p-toluenesulfonyloxy group. The compound of formula (2) can be obtained in similar epimeric purities as the compound of formula (1) including in a ratio of 7-alpha:7 beta epimers of 95:5 or higher.

Another common embodiment for the converting the compound of formula (1) into the compound of formula (A) comprises (a) oxidizing the compound of formula (1) to form an aldehyde of the formula (11):

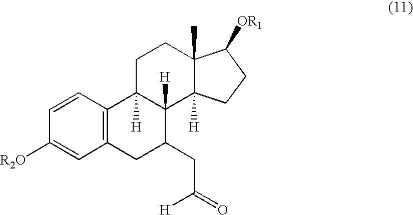

and (b) transforming the compound of formula (11) into the compound of formula (A) (in one or more steps).

Accordingly a further aspect of the invention relates to a compound of formula (11)

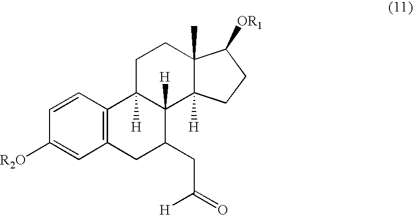

wherein R₁ and R₂ have the same meaning as above. The compound of formula (11) can be obtained in similar epimeric purities as the compound of formula (1) including in a ratio of 7-alpha:7 beta epimers of 95:5 or higher.

### DETAILED DESCRIPTION OF THE INVENTION

The present invention provides a compound of the general formula (1),

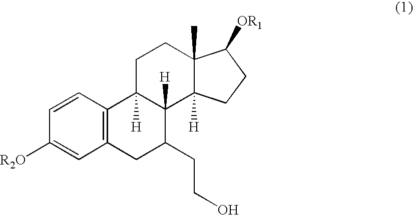

wherein R₁ is hydrogen or an acetyl group and R₂ is methyl, acetyl or benzyl group. The compound can be obtained in a solid form and, advantageously, in a crystalline state. The compound may be obtained by a suitable chemical synthesis, which is discussed in greater detail below, to obtain a crude product. The crude product generally comprises a mixture of 7-alpha and 7-beta epimers of the compound of formula (1). The word “epimer” is used because although these compounds have multiple chiral sites, they only differ on one chiral carbon. The ratio of 7-alpha to 7-beta epimers in the crude or originally synthesized product is generally less than 90:10 and typically within the range of 75:25 to 90:10, respectively. For convenience these ratios of 7-alpha to 7-beta epimers are may also expressed herein as a percentage wherein the 7-alpha epimeric purity is generally less than 90% (i.e., “less than 90:10”) and typically 75-90% (i.e., “75:25 to 90:10”). The epimeric ratio of the compound of formula (1) can be enhanced by crystallization. That is, it was discovered that the crude compound (1) may not only be crystallized from a suitable solvent to provide a solid state, preferably crystalline product, but also the difference in solubilities of both epimers in many solvents leads to the enrichment of the precipitated product by the desired 7-alpha epimer. As a result, the crystallization can provide a product comprising more than 95%, in some embodiments more than 98%, and in some embodiments even 99% or more of the 7-alpha epimer of the compound of the formula (1), based on the total amount of 7-alpha and 7-beta epimers of compound (1).

Such epimerically pure product is, indeed, a very suitable intermediate in the synthesis of fulvestrant and related compounds as the subsequent steps proceed outside the chiral centrum and the epimeric purity is generally at least maintained without any need of further epimeric purification. An advantage of the present invention is thus providing a well defined, stable crystalline intermediate in the fulvestrant synthesis having all chiral centers in the correct configuration. This avoids the need of epimeric purification in later stages of the synthesis, thus saving a lot of expensive material.

The compound of the formula (1) may be produced by one or more steps of converting a suitable steroidal precursor, e.g. nandrolon acetate, into compound of formula (1). A suitable pathway is shown in the following scheme:

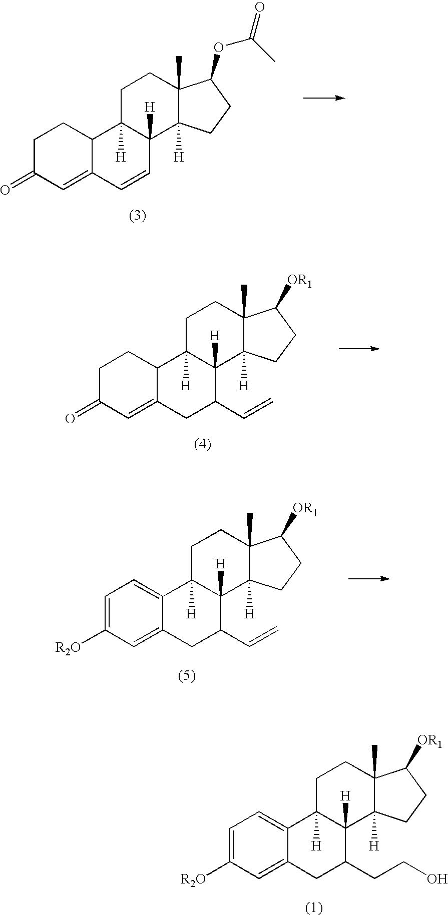

The first two steps are described in U.S. Pat. No. 6,313,108. In the first step, commercially available nandrolon acetate (3) reacts with a vinylmagnesium halide to provide acetylated 7-vinyl derivative of the formula (4), wherein R₁ is acetyl group [(17β)-17-acetyloxy-7-ethenylestr-4-ene-3-one], preferably according to a process conditions outlined in the Example 2(i) of U.S. Pat. No. 6,313,108. In such a process, a product comprising the 7-alpha/7-beta epimers of formula (4) in a ratio of approx. 8:2 to 9:1 is generally obtained.

The acetylated 7-vinyl compound may be optionally (and advantageously) deacetylated by an alkaline hydrolysis to a hydroxylated 7-vinyl derivative of the formula (4), wherein R₁ is hydrogen. An example of such process is given in the Example 2(ii) of U.S. Pat. No. 6,313,108.

Any of the above compounds of the formula (4) is then subjected to an aromatization reaction with copper (II) bromide. The aromatization reaction generally provides a compound of formula (5), where R₂ is hydrogen. This product may be O-alkylated, O-acylated or O-benzylated on the position 3 by reaction with the corresponding halide. Advantageously, the aromatization and O-alkylation may be performed in a single step. Thus, for instance, the preferred compound for making the present invention, which is the compound of formula (5) wherein R₁ is hydrogen and R₂ is methyl group [=(17β)-17-hydroxy-7-ethenyl-3-methoxyestra-1,3,5(10)-triene], may be prepared by the aromatization with copper(II) bromide in the presence of trimethylorthoformate, preferably by a process outlined in the Example 2(iii) of U.S. Pat. No. 6,313,108.

Starting from this compound of the general formula (5), the new compound of the formula (1) is prepared by a hydroboration/oxidation reaction on the vinyl double bond. A suitable hydroboration agent is a diborane or borane dimethylsulfide complex: a suitable oxidation agent is a peroxide, for instance an alkalinised solution of hydrogen peroxide. Both steps are advantageously performed sequentially, preferably without isolation of the intermediate product of the hydroboration. The hydroboration step is performed in an inert, preferably water miscible solvent, e.g. tetrahydrofuran, generally at an ambient temperature, wherein the temperature may be gradually raised up to reflux for completion of the reaction. The reaction with the peroxide advantageously proceeds also at ambient temperature. After the reaction is complete, the product is isolated by an extraction with a water-immiscible solvent, e.g. by ethyl acetate, followed by removal of the extraction solvent.

The crude product of the formula (1), which still comprises the epimeric ratio of 7-alpha to 7-beta of about 8.5:1.5, is advantageously crystallized from a solvent, which may be a C5-C10 hydrocarbon (hexane, heptane, benzene, toluene, petroleum ether etc.), C1-C6 chlorinated hydrocarbon, a C3-C10 aliphatic ester (ethyl acetate), C1-C4 aliphatic alcohol (methanol, ethanol etc.) and mixtures thereof. The crystallization is generally performed by heating the crude product in the solvent up to the reflux temperature and cooling the solution or suspension to ambient or lower than ambient temperature. Seeding the solution with a seeding crystal, partially evaporating the solvent, adding an antisolvent, and/or combinations of these techniques may be used for facilitating the crystallization. After filtration and drying, a solid, advantageously crystalline, product of the formula (1) is obtained. In general, a single crystallization of an 85% 7-alpha epimerically pure compound of formula (1) may provide a product of epimeric purity of at least 95%, often at least 98%, and in some cases at least 99% of the 7-alpha epimer. The crystallization process may be repeated, if desired or needed in order to achieve the desired range of epimeric purity. Indeed, while the above synthesis generally provides for crude product within the range of 75% to 90% 7-alpha epimeric purity, crude product having lower epimeric purity is also contemplated. Such crude product could be the result of other synthetic schemes or simply other reaction conditions or reagents. However formed, an epimerically impure 7-alpha epimer of compound (1) can be rendered more pure, i.e., “epimerically purified,” by crystallization. Achieving a desired epimeric purity can be done in one or multiple crystallizations using the same or different crystallization conditions until the desired purity is achieved.

In an alternative synthesis process, the compound (1) may be made according to the following scheme:

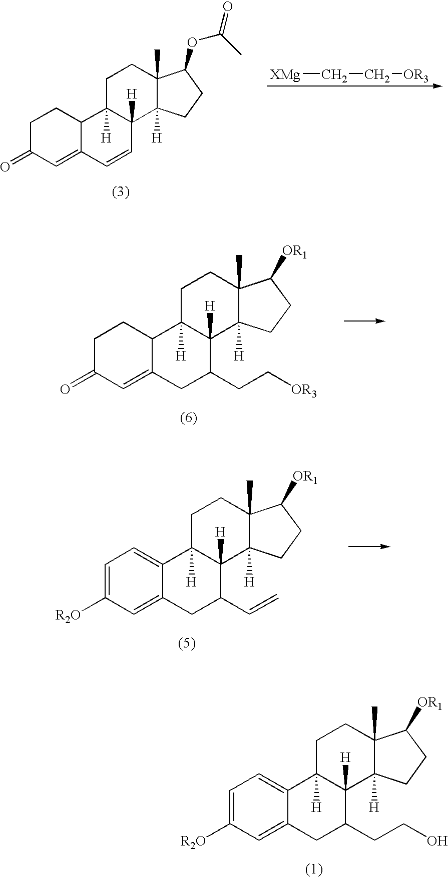

The process essentially differs from the first in that the vinylmagnesium halide is replaced by a Grignard reagent of the formula:

XMg—C₂—CH₂—OR₃,

wherein X is chloro, bromo, or iodo and R₃ is hydrogen or an O-protective group, e.g., a benzyl group. After coupling this reagent with the compound (3), the obtained alkoxyethyl-intermediate (6) is aromatised essentially as disclosed above and, optionally, the O-protective groups R₁ and R₃ are removed.

A preferred compound of the formula (1) is the compound of the formula (1a).

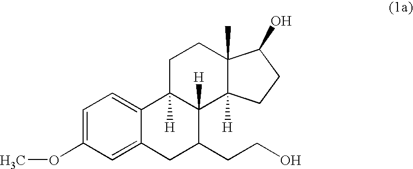

As with the compounds of formula (1), the compound of formula (1a) can be obtained as a crystalline form and can have the same high epimeric purities mentioned above, e.g., at least 95% 7-alpha epimer, etc.

The compounds of formula (1) can be used to make fulvestrant. Generally the process comprises providing a compound of formula (1) having a 7-alpha epimeric purity of at least 95% (i.e., ratio of 7-alpha epimer to 7-beta epimer in the range of 95:5 to 100:0) and converting the compound of formula (1) to a compound of the formula (A).

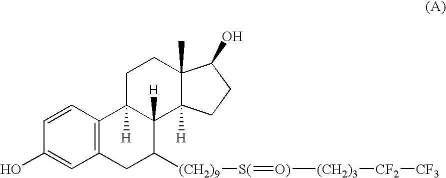

The “providing” of the compound of formula (1) in the stated epimeric purity embraces obtaining it by whatever means. Generally, the compound of formula (1) having a 7-alpha epimeric purity of at least 95% is provided by crystallizing an epimerically impure or crude compound of formula (1) as described above. Typically the epimerically impure compound (1) has a 7-alpha epimeric purity of 90% or less, often 75-90%, though less pure forms are contemplated. The crystallization improves the purity in one or more crystallization steps to at least 95%, often to at least 97%, at least 98% and even at least 99% 7-alpha epimeric purity.

The “converting” of the compound of formula (1) into a compound of formula (A) generally involves several synthetic steps. These steps are directed to forming the desired —(CH₂)₇—S(═O)—(CH₂)₃—CF₂—CF₃ group in the 7-alpha position and converting R₂ (and R₁ if it represents a non-hydrogen group) to hydrogen. It will be understood in view of the prior art that a variety of different reaction schemes can be employed by the worker skilled in the art to achieve these two chemical transformations.

A preferred process of converting the compound (1) into fulvestrant of formula (A) involves forming a compound of the formula (2)

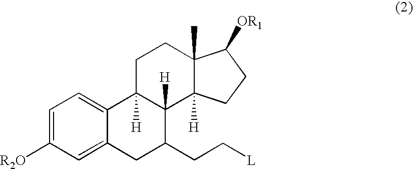

wherein L is a leaving group such as halogen, preferably bromine, an alkylsulfonyloxy group, preferably methane sulfonyloxy group, an arylsulfonyloxy group, preferably benzene sulfonyloxy- or p-toluenesulfonyloxy group. The most preferred leaving group is p-toluenesulfonyloxy group.

The conversion comprises contacting, under reactive conditions, the compound of formula (1) with a suitable donor of the leaving group (hereinafter a “donor leaving group”). For instance, the introduction of the p-toluenesulfonyloxy group can be achieved by a reaction of the compound (1) with p-toluenesulfonychloride, i.e., the donor leaving group, in a suitable inert solvent, e.g., in the presence of a base. After a conventional elaboration of the reaction mixture (neutralization and extraction of the rest of the reagents), the product may be isolated from the liquid phase and purified, if necessary.

The epimeric purity of the compound (2) is, in respect to the purity of the starting compound (1), essentially maintained. Thus, one may obtain a product comprising more than 95%, typically at least 98%, and in some embodiments at least 99% 7-alpha epimeric purity.

Some of the compounds of the general formula (2), particularly the preferred compound of the formula (2a)(L=p-toluenesulfonyloxy), may be isolated as stable solids.

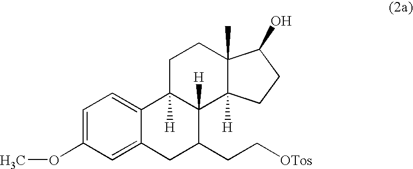

The compound of formula (2) may be transformed into fulvestrant by various processes. The common synthetic pathway comprises a suitable combination of:

a) one or more synthetic steps of converting the leaving group L of the compound (2) into the group —(CH₂)₇—S(═O)—(CH₂)₃—CF₂—CF₃; and

b) one or more synthetic steps of converting R₂ and R₁, if not hydrogen, into hydrogen;

wherein the actual order of steps may comprise whatever suitable combination of particular steps within the above process classes sub a) and sub b).

In an example of the synthetic steps of the class a), wherein the group —(CH₂)₇—S(═O)—(CH₂)₃—CF₂—CF₃ is introduced in pieces or parts, the compound (2) may first react with a Grignard compound of the formula

BrMg—(CH₂)₇—O-TBDMS

(the TBDMS is tert.butyldimethylsilyl group), to obtain a compound of the formula (8)

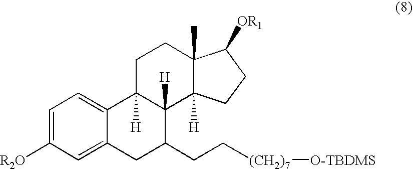

which is then converted to fulvestrant in a substantially similar way as in DE 4218743, e.g., following the conversion scheme for formula 32 in DE 4218743 to fulvestrant.

The Grignard compound is advantageously made in situ from magnesium and a bromo-compound Br—(CH₂)₇—O-TBDMS in a suitable etheral solvent (see EP 0138504) and the reaction with the compound (2) is catalysed by Li₂CuCl₄.

In another example, wherein the group —(CH₂)₇—S(═O)—(CH₂)₃—CF₂—CF₃ is introduced in a full length, the compound (2) may react with a compound of the formula

BrMg—(CH₂)₇—S—(CH₂)₃₋CF₂—CF₃

to obtain a thio-analogue of fulvestrant, which is then converted into the desired structure by an oxidation of the —S— linkage into a sulfoxy linkage.

Another option for transforming the 7-position of compound (2) into the —(CH₂)₇—S(═O)—(CH₂)₃—CF₂—CF₃ group, either in full length or in parts, involves the Suzuki reaction. The general scheme for the Suzuki reaction is as follows:

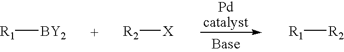

As applied to the present transformation, the compound (2) would react with an alkylborane, for instance with a compound of the formula (9)

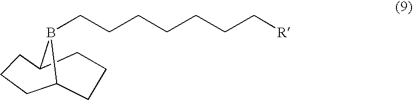

in the presence of a tetravalent palladium(0) catalyst, e.g., a catalyst made from trialkylphosphinium salt and palladium(II) salt (chloride or acetate) and a base. In an example of the per parts coupling, the R′ may be a trialkylsilyloxy group, e.g., tert.butyldimethylsilyloxy group. Alternatively, in an example of a full length coupling, the R′ is pentafluoropentylthio group.

The synthetic steps of the class b) are generally known. For example, the R₁=acetyl group may be converted into R₁=hydrogen by an alkaline hydrolysis. The R₂ group may be converted into hydrogen by proper demethylation, deacetylation or debenzylation reactions known in the art. These reactions, if necessary to be performed, may be carried out before, during, or after the sub a) class of reactions.

Another preferred process of converting the compound (1) into fulvestrant of formula (A) involves forming a compound of the formula (11)

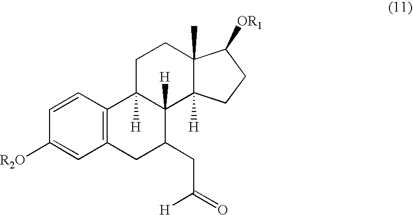

wherein R₁ and R₂ has the same meaning as in the compound (1). The conversion comprises an oxidation of the compound of formula (1) with a suitable oxidation agent in a suitable solvent. The oxidation agents include, in general, all agents that selectively oxidize primary alcohols; advantageously, such agents may comprise, e.g., TEMPO/BAIB (2,2,6,6,-tetramethyl-1-piperidineoxide and bis(acetoxy)iodobenzene combination), NMO/TPAP (N-methylmorpholine-N-oxide and tetrapropylammonium perruthenate combination), etc. The proper amounts and concentration of the oxidation agents are within the routinely used values of the prior art. The suitable solvent is a non-alcoholic organic solvent, for instance a hydrocarbon or a halogenated hydrocarbon. Advantageously, the reaction proceeds at ambient or close to ambient temperature.

The epimeric purity of the compound (11) is, in respect to the purity of the starting compound (1), essentially maintained. Thus, one may obtain a product comprising more than 95%, typically at least 98%, and in some embodiments at least 99% 7-alpha epimeric purity.

Some of the compounds of the general formula (11), particularly the preferred compound of the formula (11a)(R₁═H, R₂═CH3), may be isolated as stable solids.

The compound of formula (11) may be transformed into fulvestrant by a process which comprises reacting the compound (11) with a suitable phosphonium salt under a condition of Wittig reaction. Most preferably, the suitable phosphonium salt is a compound of the formula (12)

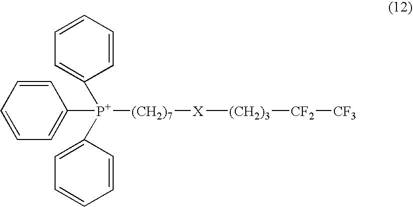

wherein X may be a —S— linkage (compound (12.1)) or a —S(═O)— linkage (compound (12.2). The reaction conditions advantageously comprise contacting, under stirring, both components in an inert solvent under a presence of a strong base. Generally, the reaction proceeds at ambient or close to ambient temperature (e.g., 20-40° C.)

The phosphonium salts of the formula (12) may be made from the corresponding halo-compounds of the formula (14)

Hal-(CH2)7-X—(CH2)3-CF2-CF3 (14)

wherein Hal- is a halo atom, preferably chlorine or bromine, and X may be a —S— linkage (compound (14.1)) or a —S(═O)— linkage (compound (14.2), by a reaction thereof with triphenylphosphine in an inert solvent. They may be used in the reaction without a need of isolation from the reaction mixture.

The product of the reaction is generally the compound of the formula (13).

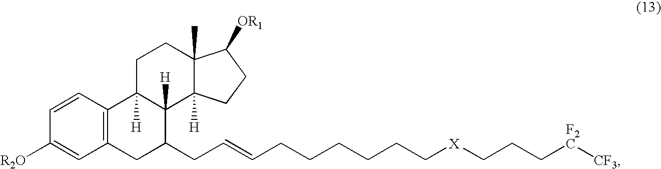

wherein X may be a —S— linkage (compound (13.1)) or a —S(═O)— linkage (compound (13.2). The epimeric purity of the compound (13) is, in respect to the purity of the starting compound (11), essentially maintained. Thus, one may obtain a product comprising more than 95%, typically at least 98%, and in some embodiments at least 99% 7-alpha epimeric purity. The configuration of the double bond of the side chain may be (E) or (Z); the actual configuration is not decisive for the future reactions.

The compound of the formula (13) may be converted into fulvestrant by a suitable combination of:

a) one or more steps of converting the group —(CH)₂—CH═CH—(CH₂)₆—X—(CH₂)₃—CF₂—CF₃ of the compound (13) into the group —(CH₂)₉—S(═O)—(CH₂)₃—CF₂—CF₃; and
b) one or more synthetic steps of converting R₂ and R₁ if not hydrogen, into hydrogen;
wherein the actual order of steps may comprise whatever suitable combination of particular steps within the above process classes sub a) and sub b).

In an example of the synthetic steps of the class a), the compound (13.1) is subjected to an oxidation reaction for to convert the —S— group into the sulfoxy-group and to a hydrogenation reaction for to convert the C═C double bond into the ethylene linkage. These steps may be carried out in any order. The oxidation reaction on the sulfur atom may be performed by contacting the —S— intermediate with, e.g., a peroxide compound, advantageously with hydrogen peroxide. The hydrogenation reaction of the double bond may be advantageously performed by a catalytic hydrogenation on, e.g. a palladium catalyst, most advantageously at an enhanced pressure of hydrogen. If the starting compound of the class a) synthetic step is the compound (13.2), then the oxidation step is not necessary. The overall product of these two steps is the compound of the formula (10) defined above.

As to the deprotection processes (Class b), they were already discussed above and apply here as well. These reactions, if necessary to be performed, may be carried out before, during, or after the sub a) class of reactions.

As the side chain conformation on the starting material of the formula (1), (2), and (11) is in the proper alpha-orientation, none of the synthetic steps leading to fulvestrant generally comprise any technique required for improving the alpha-beta ratio of the products. It is not however excluded that such technique(s), e.g. crystallization or chromatographic separation, may be applied downstream of formula (2) or (11). Nonetheless, typically such techniques when subsequently applied are intended to purify the intermediates or final product from structurally related side products and/or residual starting materials and reagents.

The invention is further illustrated by the following examples.

### Example 1

### Preparation of Compound (1)

Step 1

10.0 g of nandrolon acetate (31.8 mmole), 0.64 g Copper(I) bromide dimethylsulfide complex (3.1 mmole) and 0.28 g Lithium bromide (3.2 mmole) were stirred under nitrogen in 35 ml THF. 3.2 ml 1.0 M lithium thiophenolate solution in THF (3.2 mmole) was added via a syringe and the resulting red/brown solution was cooled to −15 to −20° C. 37.5 ml 1.7 M vinylmagnesium chloride solution in THF (63.8 mmole) was added dropwise over a period of 30 min, maintaining the temperature below −15° C. Stirring was continued for 30 min between −20 and −30° C., giving a viscous dark brown oily mixture. 25 ml saturated aqueous ammonium chloride was added dropwise, giving a less viscous mixture, containing some insoluble material. The mixture was allowed to reach room temperature and filtered over celite. The filter cake was washed with ethyl acetate. The organic layer of the filtrate was separated and concentrated in vacuo.

The residue was dissolved in 200 ml acetone and 20 ml 4 M aqueous hydrochloric acid. The resulting red solution was stirred at room temperature for 25 min. 70 ml saturated aqueous sodium bicarbonate was added until pH is 6-7. Acetone was evaporated, leaving an aqueous layer with a brown oily residue, which was extracted into ethyl acetate. The organic layer was separated and washed with brine, dried over sodium sulfate and concentrated to give 10.84 g of a brown oily residue. The oily residue was dissolved in 150 ml THF, 140 ml methanol and 45 ml water. 5.07 g potassium hydroxide was added within 5 minutes. Stirring was continued for 30 minutes at room temperature. 3.8 ml concentrated hydrochloric acid was added and stirred for 5 minutes. The mixture was concentrated and the aqueous residue extracted with 100 ml ethyl acetate. The organic extract was washed twice with brine, dried over sodium sulfate and concentrated in vacuo to give a mixture of α- and β-epimers of the compound (4) [R1=H] as brown foam (9.87 g).

Step 2

9.87 g of the compound (4) [R1=H] (crude) was stirred under nitrogen in 265 ml methanol. 12 ml trimethylorthoformate and 9.72 g copper(II) bromide were added and the mixture was heated at reflux for 1 h. After cooling to room temperature again, the mixture was filtered. The filtrate was concentrated and the residue dissolved in ethyl acetate. The solution was washed with saturated aqueous sodium bicarbonate. The washing layer was back-extracted once with ethyl acetate and this extract washed with saturated aqueous sodium bicarbonate and brine. The organic layers were combined were washed with brine, dried over sodium sulfate and concentrated to give a solid foam (9.22 g). The crude product was purified by eluting over silica gel with heptane/ethyl acetate 3/2. The compound (5) [R1=H, R2=methyl] was thus obtained in 4.55 g yield.

Step 3

4.55 g of the compound (5) [R1=H, R2=methyl] was stirred under nitrogen in 50 ml THF. To the solution was added via syringe 1.23 g borane dimethylsulfide complex. The temperature increased and the colour changed from orange/yellow into yellow. After 30 minutes a gel formed. The mixture was diluted with 25 ml THF and the mixture was heated to reflux, giving a stirrable suspension. After one hour reaction was complete and the mixture was cooled to 15-20° C. A solution of 1.17 g sodium hydroxide in 10 ml water followed by 3.75 ml 35 wt % hydrogen peroxide was added. After 30 minutes the mixture was diluted with ethyl acetate and water. The layers were allowed to separate overnight. Organic layer was separated and set aside. The aqueous layer was extracted with ethyl acetate. The combined organic layers were washed with brine, dried over sodium sulfate and concentrated to give an off-white to yellow foam (4.74 g) of the compound (1) [R1=H, R2=methyl].

Crystallization

The foam was stirred in a mixture of 10 ml heptane and 14 ml ethyl acetate and heated. Without completely dissolving, the foam changed into a white crystalline solid. The crystals were isolated by filtration, yielding 1.3 g of epimerically pure 1.

### Example 1a

### Preparation of the Compound (1a)

In a 250 ml three-necked flask, compound (5) (5.38 g, 17.22 mmol) was stirred under nitrogen at room temperature in THF (50 ml) to give a yellow solution. To the clear solution was added borane dimethyl sulfide complex (1.85 ml, 18.31 mmol) to give a slightly yellow solution, while gas evolved from the solution. Reaction temperature increased to 33° C. After 2 minutes stirring, the yellow solution became more viscous and became a gel. The mixture was diluted with THF (50 ml) and heated to 40° C. The gel disintegrated and a colorless solution with white precipitate formed. The mixture was further heated to reflux and kept at reflux for 30 minutes. The reaction mixture was then allowed to cool to room temperature (rt) and further cooled in an ice bath to 5° C. A solution of sodium hydroxide (1.547 g, 38.7 mmol) in water (10 ml) was added (T_{R}→10° C.), followed by portionwise addition of hydrogen peroxide (4.63 ml, 52.9 mmol) while maintaining the reaction temperature below 15° C. After complete addition, stirring was continued for 80 min while cooling in ice. HPLC after 20 minutes showed a complete reaction. A white solid that had precipitated, was removed by decantation. The resulting colorless solution was concentrated and dissolved in ethyl acetate (75 ml) and water (35 ml) and shaken vigorously. The layers were allowed to separate and a white solid crystallized in the upper organic layer. The two-phase system was warmed up a little bit to dissolve the solids. The aqueous layer was separated and extracted once with warm ethyl acetate (50 ml). The combined organic layers contained a white precipitate and were heated until a clear almost colorless solution was obtained. The solution was allowed to cool down again while stirring in an ice bath. A white solid crystallized. After 2 h, the suspension formed was filtered over a glass filter, yielding a white crystalline solid (3.73 g; 91% purity (area % HPLC)). The mother liquid contained less pure product (2.7 g; 69% purity (area % HPLC)).

The crystalline solid was recrystallized from ethyl acetate (25 ml) and yielded the alpha-form of the compound (1a) (2.15 g, 6.51 mmol, 37.8% yield) with a purity of 96% (3.5% of beta form).

From the remaining mother liquids a second crop of product of similar purity was isolated by recrystallizations (0.45 g). Total yield: 2.6 g (46%). NMR confirmed the alpha-isomer of alcohol (1a).

### Example 2

### Preparation of the Compound (2)

1.09 g of the compound (1) from Example 1 was stirred in 25 ml dichloromethane (not completely dissolved) 2.31 ml triethylamine was added followed by 0.69 g tosylchloride. Stirring was continued overnight at room temperature. The reaction mixture was washed twice with 20 ml 1 M hydrochloric acid and then once with 20 ml saturated aqueous sodium bicarbonate. The solution was dried over sodium sulfate and concentrated to give the tosylate (2) as a white solid (1.52 g; 95%).

### Example 3

### Preparation of Compound (8)

180 mg of magnesium turnings were suspended in 10 ml of sodium dried THF. 1.9 g of (7-Bromo-heptyloxy)-tert-butyl-dimethyl-silane was slowly added, during addition, brown colour disappeared. After 1 hour of reflux, the reaction mixture was cooled to −40° C. 0.75 g of the tosylate of the Example 2 in 5 ml of dry THF was added. 2 ml of 0.1 M Li₂CuCl₄ in THF was added at −40-−35° C. After addition, the reaction mixture was allowed to heat up to room temperature. During warming up the mixture changed from a tan suspension into a clear brown solution. The brown colour becomes darker. After 30 minutes, the reaction mixture became dark purple to black. After 1 night, the reaction mixture was quenched with 10 ml of NH₄Cl solution.

10 ml of diethyl ether was added. The rests of Magnesium were filtered off. The layers were separated. The organic layer was washed with 10 ml of saturated NaHCO₃ solution, dried on Na₂SO₄ and concentrated in vacuo. 1.7 g of an oil was obtained. An analytical sample was purified twice over Silica 1) 35% EtOAc in Heptane; 2) 20% EtOAc in Heptane.

### Example 4

### Preparation of Thioanalogue of Fulvestrant of the Formula (16) [R₁ is Hydrogen and R₂ is Methyl]

Magnesium turnings (150 mg) were stirred under nitrogen in THF (10 ml) and a crystal of iodine was added. Gentle reflux started. A syringe was filled with bromothioether (compound (14.1(Hal- is Br) (1.78 g) and half of it was added. When the reaction started, the rest of the bromothioether was added. The mixture was allowed to cool to room temperature and kept overnight.

Another flask under nitrogen atmosphere was charged with the tosylate compound (2a) (250 mg) in THF (4 ml) and cooled to −40° C. After stirring with one equivalent methyl magnesium bromide, a 0.1M solution of Li₂CuCl₄ in THF (0.5 ml) was added giving an orange solution. The Grignard solution prepared above (7 ml) was added carefully via syringe. The color changed via green within a few seconds into orange/red. After complete addition, the mixture was allowed to reach room temperature, resulting in a darker color of the mixture. The tosylate had disappeared after 4 days and the mixture was quenched with saturated aqueous ammonium chloride and diluted with diethyl ether. The organic layer was isolated, dried over sodium sulphate and concentrated to give a brown oily residue (1.16 g). The mixture was purified by flash column chromatography (heptane/ethyl acetate 4/1). The product was isolated (100 mg) and its structure was confirmed by NMR.

The thioether compound can be converted into fulvestrant by a deprotection of the methyl group (converting R² from methyl into hydrogen) and an oxidation of the thioether linkage to the sulfoxide in any order.

### Example 5

### Preparation of compound (11a)

In a 50 mL round-bottomed flask, the compound (1a) (0.82 g, 2.481 mmol) and 2,2,6,6,-tetramethyl-1-piperidineoxide [TEMPO] (40 mg, 0.256 mmol) were stirred at room temperature in dichloromethane (7 ml) to give a yellow suspension. To the resulting suspension was added iodobenzene diacetate (BAIB) (0.88 g, 2.73 mmol) to give a yellow suspension. Stirring was continued at room temperature. After a while, a yellow solution was obtained. HPLC after 1.5 hour showed almost disappearance of the alcohol (<1.5%). After 2 h, the mixture was diluted with dichloromethane (10 ml) and washed with aqueous sodium thiosulfate. The aqueous layer was extracted once with dichloromethane. The combined organic layers were washed with sat.aq.bicarbonate, brine, dried (sodium sulfate) and concentrated to give an orange oily residue (1.44 g).

The crude mixture was purified by column chromatography (heptane:ethyl acetate/2:1), giving the desired aldehyde (11a) (0.46 g, 1.401 mmol, 56.4% yield) as a white solid.

### Example 5a

### Preparation of Compound (11a)

In a 100 mL round-bottomed flask, the compound (1a) (2.52 g, 7.63 mmol) and 2,2,6,6,-tetramethyl-1-piperidineoxide [TEMPO] (0.12 g, 0.768 mmol) were stirred at room temperature in dichloromethane (20 ml) to give a pale orange suspension. To the resulting mixture was added portionwise iodobenzene diacetate (BAIB) (2.70 g, 8.39 mmol). Stirring was continued at rt for 2 hr, during which time the suspension changed into a clear orange solution. HPLC after 1.25 hr and TLC after 2 hr showed that the alcohol had been converted. The mixture was diluted with aqueous sodium thiosulfate and vigorously stirred. The aqueous layer was extracted once with dichloromethane. The combined organic layers were washed with sat.aq.bicarbonate (2×), brine, dried (sodium sulfate) and concentrated to give an orange oily residue (4.18 g).

The crude mixture was purified by column chromatography (Reveleris pre-packed column 40 g silica; heptane:ethyl acetate/3:1; 35 ml/min; fractions of 20 s). In total, the desired aldehyde (11a) (1.44 g, 4.38 mmol, 57.5% yield) was obtained from fractions 40-70. The structure and epimeric purity was confirmed by NMR.

### Example 6

### Preparation of Compound (13.1) [R1=H, R2=CH3]

Step 1

In a 50 mL three-necked flask, (7-bromoheptyl)(4,4,5,5,5-pentafluoropentyl)sulfane (1.97 g, 5.31 mmol) was stirred under nitrogen at room temperature in toluene (6 ml) to give a colorless solution. To the clear solution was added triphenylphosphine (1.4 g, 5.34 mmol). The mixture was heated at reflux for 19 hr. A white suspension had formed. Toluene (40 ml) was added and 20 ml of the solvents were distilled off (to remove traces of water azeotropically). The mixture was allowed to cool to room temperature and then further cooled in an ice bath.

Step 2

3.3 ml of a 1.6 molar solution of n-butyllithium in heptane was added to the product of Step 1, resulting in an orange solution. Stirring was continued for 1 h while cooling. A solution of the compound (11a) (1.44 g, 4.38 mmol) in toluene (10 ml) was added via syringe. The deep orange color disappeared and a tan suspension was obtained. Stirring was continued at rt. TLCs after 1.5 h and 3 hr showed formation of the coupled product; there is still starting aldehyde present. After stirring for 5 days, TLC showed only a weak spot left of the starting aldehyde. Water (20 ml) was added to the mixture, followed by vigorous stirring. A three-phase system was obtained. The organic layer was separated from the lower aqueous layer containing a brown oily residue. The aqueous layer was extracted twice with ethyl acetate. The combined ethyl acetate layers were combined with the toluene layer, washed with brine, dried (sodium sulfate) and concentrated to give a red/brown oily residue (4.21 g). The oily residue was purified by column chromatography (heptane:ethyl acetate/3:1, reveleris column 40 g silica; 30 ml/min), yielding the product (13.1) [R1=H, R2=CH3] (0.54 g, 0.896 mmol, 21% yield).

### Example 7

### Preparation of Compound (13.2) [R1=H, R2=CH3]

In a 25 mL round-bottomed flask, the compound (13.1) [R1=H, R2=CH3] (500 mg, 0.829 mmol) was stirred at room temperature in ethyl acetate (10 ml) to give a colorless solution. Acetic acid (0.29 ml, 5.02 mmol) and hydrogen peroxide (0.14 ml, 1.635 mmol) were added. The clear solution was stirred at 21° C. for 48 hr. A solution of sodium sulphite (0.6 g) in water (10 ml) was added and stirring was continued vigorously for 15 minutes. The mixture was neutralized with 1 M aqueous NaOH (5 ml). The aqueous layer was separated and extracted with ethyl acetate. The combined organic layers were washed with water, dried (sodium sulfate) and concentrated to give an almost colorless oily residue (588 mg).

The crude mixture was purified by column chromatography (Reveleris column 12 g; heptane:ethyl acetate/1/1), yielding the desired product (13.2) (354 mg, 0.572 mmol, 69.0% yield).

### Example 8

### Preparation of Compound (10) [R1=H, R2=CH3]

The autoclave was charged with the compound (13.2) [R1=H, R2=CH3] (110 mg, 0.178 mmol) and ethyl acetate (5 ml). Nitrogen was bubbled through the solution and palladium/carbon 10% (90 mg, 0.085 mmol) was added. The autoclave was closed and filled with hydrogen (0.358 mg, 0.178 mmol) until a pressure of 50 bar was reached. Stirring was continued at 21° C. for 2 hr. HPLC after 2 h showed complete conversion. The reaction mixture was filtered over celite and then concentrated to obtain the compound (10) [R1=H, R2=CH3] (87 mg, 0.140 mmol, 79% yield).

### Example 9

### Preparation of Fulvestrant

A solution of aluminum trichloride (218 mg, 1.631 mmol) and thiourea (93 mg, 1.224 mmol) in dichloromethane (2 ml) (prepared by adding 0.47 g thiourea in portions to a solution of 1.1 g aluminum trichloride in 10 ml dichloromethane, stirring for 15 minutes and then taking 2 ml of the resulting solution.) was stirred at rt. To the clear solution was added a solution of compound (10) [R1=H, R2=CH3] (80 mg, 0.129 mmol) in dichloromethane (5 ml). Stirring was continued at 55° C. for 22 hr. The reaction mixture was diluted with dichloromethane and water and stirring was continued for 2 hr. The combined organic layers were dried (sodium sulfate) and concentrated to give an almost colorless oily residue (58 mg). Fulvestrant (25 mg, 0.041 mmol, 32.0% yield) was isolated from the mixture by flash column chromatography: Reveleris column (12 g); heptane/ethyl acetate 1/1 40 fr.; heptane/ethyl acetate 1/2 40 fr.; 25 ml/min; fractions of 20 s; fractions 50-68 combined. NMR confirmed the structure.

### Example 10

### Compound (11a)

In a 50 mL round-bottomed flask, diastereomerically pure (7S,13S)-7-(2-hydroxyethyl)-3-methoxy-13-methyl-7,8,9,11,12,13,14,15,16,17-decahydro-6H-cyclopenta[a]phenanthren-17-ol [compound 1a] (1.0 g, 3.03 mmol) and TEMPO (46 mg, 0.294 mmol) were stirred at room temperature in acetic Acid (10 ml). To the resulting mixture (alcohol not completely dissolved) was added iodobenzene diacetate (BAIB) (1.07 g, 3.32 mmol) in portions over a period of one minute. The added BAIB dissolved slowly, giving a yellow to orange solution. Stirring was continued at rt for 24 hr. HPLC after 6 hr showed 91% conversion. After leaving the reaction overnight, conversion was nearly complete. The reaction mixture was poured into ice/water and stirred. A milky suspension was obtained with some yellow sticky solid. The mixture was allowed to reach rt and stirring was continued. Acetic acid (approx. 5 ml) was added and stirring was continued while allowing to cool to rt. The solids were filtered off and washed twice with diethyl ether, giving a white solid (0.91 g). The solid was dried in vacuo at 40° C. for 16 hr, giving 2-((7S,13S)-17-hydroxy-3-methoxy-13-methyl-7,8,9,11,12,13,14,15,16,17-decahydro-6H-cyclopenta[a]phenanthren-7-yl)acetaldehyde (0.67 g, 2.040 mmol, 67% yield) as a white solid. ¹H-NMR confirmed the structure.

### Example 11

### Preparation of compound (13.2) [R1=H, R2=CH3]

In a 25 mL round-bottomed flask, (7R,13S)-3-methoxy-13-methyl-7-((E)-9-(4,4,5,5,5-pentafluoropentylthio)non-2-enyl)-7,8,9,11,12,13,14,15,16,17-decahydro-6H-cyclopenta[a]phenanthren-17-ol [compound 13.1] (320 mg, 0.531 mmol) was stirred at room temperature in ethyl acetate (10 ml) to give a colorless solution. Acetic acid (0.184 ml, 3.19 mmol) and hydrogen peroxide (0.091 ml, 1.062 mmol) were added. The clear solution was stirred at 21° C. for 94 hr. A solution of sodium sulphite (0.6 g) in water (10 ml) was added and stirring was continued vigorously for 15 minutes. The aqueous layer was separated and extracted with ethyl acetate. The combined organic layers were washed with water, dried (sodium sulphate) and concentrated to give an almost colorless oily residue (339 mg).

The crude mixture was purified by column chromatography (heptane/ethyl acetate 40/60), yielding (7R,13S)-3-methoxy-13-methyl-7-((E)-9-(4,4,5,5,5-pentafluoropentylsulfinyl)non-2-enyl)-7,8,9,11,12,13,14,15,16,17-decahydro-6H-cyclopenta[a]phenanthren-17-ol (214 mg, 0.346 mmol, 65% yield)

### Example 12

### Preparation of Intermediate 10 (R1=H, R2=CH3)

The autoclave was charged with (7R,13S)-3-methoxy-13-methyl-7-((E)-9-(4,4,5,5,5-pentafluoropentylsulfinyl)non-2-enyl)-7,8,9,11,12,13,14,15,16,17-decahydro-6H-cyclopenta[a]phenanthren-17-ol [compound 13.2] (200 mg, 0.323 mmol) and ethyl acetate (10 ml). Nitrogen was bubbled through the solution and palladium/carbon 10% (210 mg, 0.197 mmol) was added. The autoclave was closed and filled with hydrogen until a pressure of 40 bars was reached. Stirring was continued at 21° C. for 1.5 hr.

TLC showed complete conversion. The reaction mixture was filtered over celite, washed with ethyl acetate and then concentrated to give (7R,13S)-3-methoxy-13-methyl-7-(9-(4,4,5,5,5-pentafluoropentylsulfinyl)nonyl)-7,8,9,11,12,13,14,15,16,17-decahydro-6H-cyclopenta[a]phenanthren-17-ol (178 mg, 0.287 mmol, 89% yield).

### Example 13

### Preparation of Fulvestrant

A solution of aluminum trichloride (427 mg, 3.20 mmol) and thiourea (183 mg, 2.403 mmol) in dichloromethane (3.9 ml) (prepared by adding 0.47 g thiourea in portions to a solution of 1.1 g aluminum trichloride in 10 ml dichloromethane, stirring for 15 minutes and then taking 3.9 ml of the resulting solution.) was stirred at rt. To the clear solution was added a solution of methoxyfulvestrant [intermediate 10 (R1=H, R2=CH3)] (157 mg, 0.253 mmol) in dichloromethane (10 ml). Stirring was continued at 38° C. for 22 hr. After 2 hr, HPLC showed 6% conversion of the starting material into fulvestrant in a further clean reaction. HPLC after 22 hr showed complete conversion. The reaction mixture was diluted with dichloromethane and water and stirring was continued for 2 hr. The aqueous phase was isolated and extracted with dichloromethane. The combined organic layers were dried (sodium sulfate) and concentrated to give an almost colorless oily residue (101 mg). Fulvestrant (42 mg, 0.069 mmol, 27.4% yield) was isolated from the mixture by flash column chromatography (heptane/ethyl acetate 40/60). NMR showed fulvestrant pure in the desired 7α-configuration.

Each of the patents, patent applications, and journal articles mentioned above are incorporated herein by reference. The invention having been described it will be obvious that the same may be varied in many ways and all such modifications are contemplated as being within the scope of the invention as defined by the following claims.

We claim:

1. A compound of formula (1) 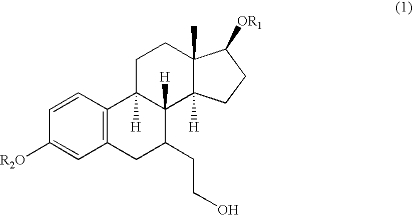
wherein R₁ represents hydrogen or an acetyl group and R₂ represents a methyl, acetyl, or benzyl group.

2. The compound according to claim 1, wherein said compound has a ratio of 7-alpha epimer to 7-beta epimer in the range of 95:5 to 100:0.

3. The compound according to claim 1, in the form of a crystalline material.

4. The compound according to claim 3, wherein R₁ is hydrogen and R₂ is methyl.

5. The compound according to claim 4, wherein said compound has a ratio of 7-alpha epimer to 7-beta epimer in the range of about 99:1 to 100:0.

6. A compound of formula (2) or (11): 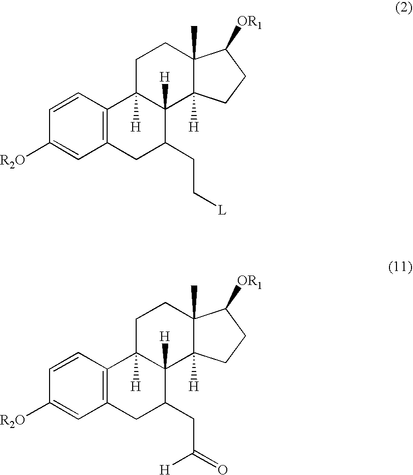
wherein R₁ represents hydrogen or an acetyl group; R₂ represents a methyl, acetyl, or benzyl group; and L represents a leaving group.

7. The compound according to claim 6, wherein said compound has a ratio of 7-alpha epimer to 7-beta epimer in the range of 95:5 to 100:0.

8. A process, which comprises crystallizing an epimerically impure compound of formula (1): 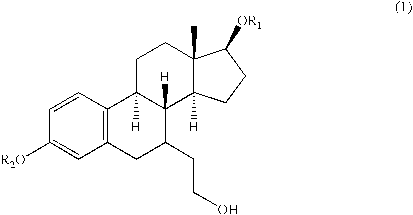
wherein R₁ represents hydrogen or an acetyl group and R₂ represents a methyl, acetyl, or benzyl group, to form an epimerically purified compound of formula (1) in the form of a crystalline material.

9. The process according to claim 8, wherein said epimerically purified compound of formula (1) has a 7-alpha epimeric purity of at least 95%.

10. The process according to claim 9, wherein said epimerically impure compound of formula (1) has a 7-alpha epimeric purity in the range of 75% to 90%.

11. A process for making fulvestrant, which comprises:
(i) providing a compound of formula (1) having a 7-alpha epimeric purity of at least 95% 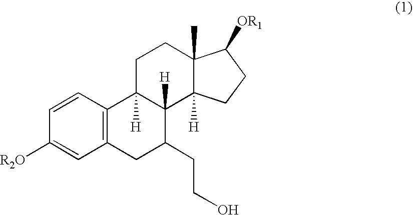
wherein R₁ represents hydrogen or an acetyl group and R₂ represents a methyl, acetyl, or benzyl group; and
(ii) converting said compound of formula (1) to a compound of the formula (A)

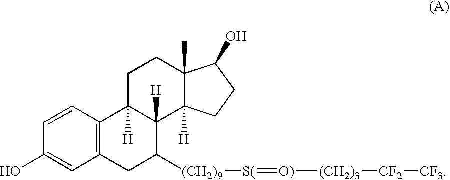

12. The process according to claim 11, wherein said providing step (i) comprises crystallizing an epimerically impure compound of formula (1) having a 7-alpha epimeric purity of 90% or less, to obtain said compound of formula (1) having said 95% epimeric purity.

13. The process according to claim 11, wherein said providing step (i) provides a compound of formula (1) having a 7-alpha epimeric purity of at least 99%.

14. The process according to claim 11, wherein said converting step (ii) comprises:
(a) reacting said compound of formula (1) with a donor leaving group to form a compound of formula (2) 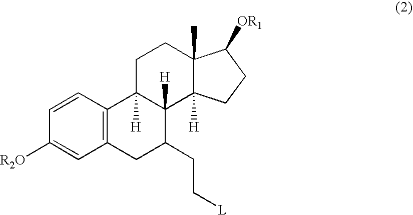
wherein L represents a leaving group; and
(b) transforming said compound of formula (2) into said compound of formula (A).

15. The process according to claim 14, wherein said transforming step (ii)(b) comprises reacting said compound of formula (2) with a Grignard reagent of the formula
BrMg—(CH₂)₇—O-TBDMS
to form a compound of formula (8) 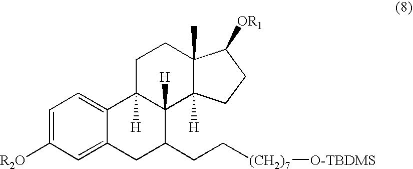
and subsequently converting said compound of formula (8) into said compound of formula (A).

16. The process according to claim 14, wherein said transforming step (ii)(b) comprises reacting said compound of formula (2) with a Grignard reagent of the formula
BrMg—(CH₂)₇—S—(CH₂)₃₋CF₂—CF₃
and subsequently oxidizing the thio linkage to form a sulfoxy linkage.

17. The process according to claim 14, wherein L represents a halogen or a sulfonyloxy group.

18. The process according to claim 14, wherein said transforming step (ii)(b) comprises converting R₂ into hydrogen.

19. The process according to claim 11, wherein said converting step (ii) comprises:
(a) oxidizing said compound of formula (1) to form an aldehyde of the formula (11) 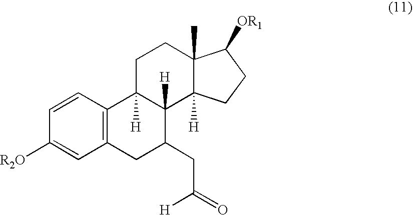
and (b) transforming the compound of formula (11) into the compound of formula (A).

20. The process according to claim 19, wherein the transforming step (b) comprises reacting the compound of formula (11) with a compound of the formula (12) 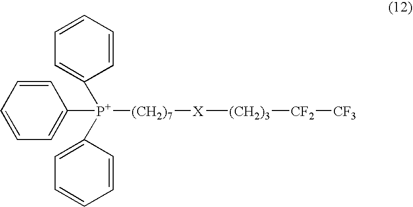
wherein X is a —S— linkage or a —S(═O)— linkage, to form a compound of formula (13) 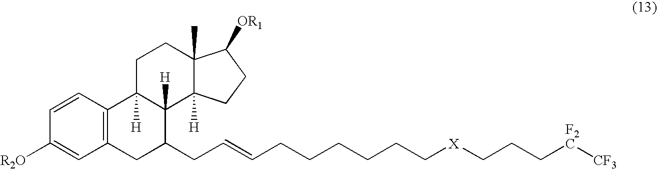
and hydrogenating the double bond in the compound of formula (13).

21. The process according to claim 20, wherein X represents a sulfide linkage and said transforming further comprises oxidizing the sulfide linkage to form a sulfoxide linkage.

22. The process according to claim 19, wherein said transforming step (ii)(b) comprises converting R₂ into hydrogen.
